(19) United States
(12) Patent Application Publication
(10) Pub. No.: US 2024/0180959 A1
(43) Pub. Date: Jun. 6, 2024

(54) USE OF A COMPOSITE MATERIAL

(71) Applicant: Heraeus Medical GmbH, Wehrheim (DE)

(72) Inventors: Sebastian VOGT, Wehrheim (DE); Thomas KLUGE, Wehrheim (DE)

(73) Assignee: Heraeus Medical GmbH, Wehrheim (DE)

(21) Appl. No.: 18/529,500

(22) Filed: Dec. 5, 2023

(30) Foreign Application Priority Data
Dec. 6, 2022 (EP) 22211559.4

(51) Int. Cl. A61K 33/10; A61K 31/7036; A61K 33/06; A61P 19/08

(52) U.S. Cl. CPC A61K 33/10; A61K 31/7036; A61K 33/06; A61P 19/08

(57) ABSTRACT

One aspect relates to a composition for use in a therapeutic procedure, which comprises calcium sulfate, an alkaline earth carbonate, a binder, and up to 2.5 percent by weight gentamicin sulfate, based on the total mass of the composition, and to a production method for such a composition. The composition can be used as a bone substitute material.

### CROSS-REFERENCE OF RELATED APPLICATION

This Utility Patent application claims priority to European Patent Application No. 22211559.4, filed Dec. 6, 2022, which is incorporated herein by reference.

### TECHNICAL FIELD

One aspect relates to the field of orthopedics, in particular composite materials for use as bone substitute material.

### TECHNICAL BACKGROUND

Bone substitute materials can be used to fill bone cavities. In principle, this can be done with the patient's own tissue which is well tolerated by the patient but can often only be obtained in a limited amount. In practice, therefore, artificial materials must often be used as bone substitute materials for larger bone cavities. Calcium sulfate-containing bone substitute materials are described, for example, in patent documents US2002110541A, U.S. Pat. No. 5,807,567A, US2002197315A, U.S. Pat. Nos. 6,652,887A, 5,756,127A, 5,614,206A and DE19953771C. In surgery, there is in principle the risk of microbial infection occurring as a result of the medical intervention, for example as a result of microbially loaded bone substitute materials or medical instruments. In other cases, an infection is already present before surgery, and there is a risk that the pathogens causing the infection cannot be completely removed from the infected tissue during surgery.

### EMBODIMENTS

The present application describes bone substitute materials and the production thereof. The bone substitute materials can contain an antibiotic which is preferably released in a delayed manner from the bone substitute materials. Furthermore, therapeutic applications of bone substitute materials are described.

Embodiments of the invention are described below.

In a first embodiment, a first aspect relates to a composition for use in a therapeutic procedure, wherein the composition comprises calcium sulfate, an alkaline earth carbonate, a binder, and more than 0 to at most 2.5 percent by weight gentamicin sulfate, preferably 0.5 to 2.5 percent by weight gentamicin sulfate, based on the total mass of the composition.

In a second embodiment, the use comprises mixing the composition with viable cells in vitro, wherein the mixing preferably causes a homogeneous mixture to be formed.

In a third embodiment of the composition for use according to the second embodiment, the viable cells, in the form of blood, preferably whole blood, bone marrow concentrate, and/or in the form of cancellous bone, are mixed in vitro with the composition.

In a fourth embodiment of the composition for use according to the third embodiment, the cancellous bone is an autologous cancellous bone or an allogeneic cancellous bone.

In a fifth embodiment of the composition for use according to embodiment 3 or 4, the cancellous bone is present in the form of bone chips.

In a sixth embodiment of the composition for use according to the fifth embodiment, the bone chips have a mean diameter of 1 to 10 mm.

In a seventh embodiment of the composition for use according to any of embodiments 3 to 6, the composition is mixed with cancellous bone or bone marrow concentrate in vitro in a weight ratio of “1 to 0.5” to “1 to 3.”

In an eighth embodiment of the composition for use according to the third embodiment, the composition is mixed with blood in vitro in a weight ratio of “1 to 0.2” to “1 to 1.”

In a ninth embodiment of the composition for use according to any of embodiments 3 to 8, a part by weight of the composition is mixed in vitro with 0.5 to 3.0 parts by weight cancellous bone or bone marrow concentrate and with 0.2 to 0.8 parts by weight blood.

In a tenth embodiment of the composition for use according to any of embodiments 3 to 9, the blood is subjected to coagulation after being mixed with the composition.

In an eleventh embodiment of the composition for use according to any of the preceding embodiments, the use comprises introducing the composition into a bone defect, for example a bone cavity.

In a twelfth embodiment of the composition for use according to any of the preceding embodiments, the composition comprises 70 to 80 percent by weight calcium sulfate.

In a thirteenth embodiment of the composition for use according to any of the preceding embodiments, the composition comprises 12 to 18 percent by weight alkaline earth carbonate.

In a fourteenth embodiment of the composition for use according to any of the preceding embodiments, the composition comprises 7 to 12 percent by weight binder.

In a fifteenth embodiment of the composition for use according to any of the preceding embodiments, the therapeutic procedure comprises the filling of a bone defect, caused by a trauma, tumor or infection, with the composition.

A further aspect relates to a method for producing a composition, comprising the following steps:
grinding calcium sulfate, an alkaline earth metal carbonate, a binder, more than 0 to at most 2.5 percent by weight gentamicin sulfate, preferably 0.5 to 2.5 percent by weight gentamicin sulfate, to a powder:
compacting the powder to form an agglomerate:
breaking down the agglomerate to a granulate; and
fractionating the granulate to a desired particle size.

### DETAILED DESCRIPTION

In principle, for the embodiments described herein, the elements of which “contain” or “comprise” a particular feature (e.g., a material), a further embodiment is always considered in which the element in question consists of that feature alone, i.e., comprises no further components. The word “comprise” or “comprising” is used herein synonymously with the word “contain” or “containing.”

If an element is referred to in the singular in an embodiment, an embodiment is also considered in which a plurality of these elements are present. The use of a term for an element in the plural fundamentally also encompasses an embodiment in which only a single corresponding element is contained.

Unless otherwise indicated or clearly precluded by the context, it is possible in principle, and is herewith clearly taken into consideration, that features of different embodiments may also be present in the other embodiments described herein. It is also considered in principle that all features that are described herein in conjunction with a product, for example a composition, are also applicable to the uses described herein of such a product, and vice versa. All such considered combinations are not explicitly listed in all instances, simply to keep the description brief. Technical solutions which are known to be equivalent to the features described herein are also intended to be encompassed in principle by the scope of the invention.

One aspect of the invention relates to a composition for use in a therapeutic procedure and comprising calcium sulfate, an alkaline earth carbonate, a binder, and gentamicin sulfate. The composition preferably comprises more than 0 and at most 2.5 percent by weight gentamicin sulfate, based on the total mass of the composition.

In one embodiment, the composition comprises 0.5 to 2.5 percent by weight gentamicin sulfate, based on the total mass of the composition. In one embodiment, the composition comprises 1.2 to 2.0 percent by weight gentamicin sulfate. In one embodiment, the composition comprises 1.4 to 1.8 percent by weight gentamicin sulfate. In one embodiment, the composition comprises 1.5 to 1.7 percent by weight gentamicin sulfate, or 1.55 to 1.65 percent by weight, based on the total mass of the composition. In some embodiments, the composition comprises an effective amount of gentamicin sulfate comprising at most 2.5 percent by weight gentamicin sulfate, based on the total mass of the composition.

Gentamicin is an aminoglycoside complex which can be produced by fermenting Micromonospora purpurea or Micromonospora echinospora. In its action as an antibiotic, it is usually used as a sulfate salt, the gentamicin sulfate. By binding to the 30S ribosome subunit, gentamicin causes the erroneous reading of the codons by blocking the translocation of the peptidyl-tRNA from the acceptor side to the donor side.

An “effective amount” of gentamicin sulfate may be an amount sufficient to provide a composition described herein which reduces at least the growth rate of microbial organisms in the range of the composition compared to a control. In many embodiments, the amount of gentamicin sulfate is sufficient to produce an inhibition zone having a diameter of at least about 10 mm, typically at least approximately 15 mm, measured using the antibiotic activity test according to U.S. Pat. No. 5,968,253.

In some embodiments, a composition according to the invention contains an amount of gentamicin sulfate that is high enough to develop an antimicrobial effect in the patient but is sufficiently low so as not to cause any cytotoxic effects.

In one embodiment, the composition comprises particles which contain the above-mentioned components, namely calcium sulfate, an alkaline earth carbonate, a binder and gentamicin sulfate. Such a composition is also referred to herein as “granulate,” and the individual particles are also referred to as “granules.” Preferably, the calcium sulfate, the alkaline earth carbonate, the binder and the gentamicin sulfate are present together in the particles. This means that the particles each comprise a mixture of the stated components. It is preferably a homogeneous mixture in which the stated components, in particular the calcium sulfate, the alkaline earth carbonate, and the gentamicin sulfate are distributed uniformly.

The particles can have an irregular shape. This means that the particles in particular do not have a perfect spherical or ellipsoidal geometry but have irregular edges on their surface. Such particles can be produced, for example, by breaking down agglomerates. Such an irregular shape facilitates the bonding of the particles to one another, optionally to further components, for example for filling a bone cavity.

Such irregularly shaped particles may have advantageous release properties with respect to the calcium and/or gentamicin contained therein. As a result, a better efficacy and/or agreeability of the composition can be achieved. Without wishing to be bound to any theory, it is believed that these modified properties can be achieved due to the modified surface properties of such granules compared to regularly shaped, for example cylindrically or spherically pressed, tablets.

A bone defect, which comprises a cavity in a bone, is referred to herein as a “bone cavity.” Such a bone cavity can be formed, for example, due to injury (also referred to in the art as “trauma”), by infection-related inflammation, or due to another disease of the bone. A surgical intervention, such as a debridement, can also cause bone cavities and other bone defects. “Agglomerates” are understood herein to mean, in particular, forms which can be produced by compacting or pressing powders. An example of an agglomerate is a tablet produced by pressing a powder.

In some embodiments, the particles have a mean diameter of 1.0 to 7.0 mm. In some embodiments, the mean diameter of the particles is 0.5 to 10 mm, 1 to 3 mm, 2 to 6 mm, 3 to 5 mm, or 3.5 to 4.5 mm. This mean diameter of the particles can be determined by fractional screening, as is customary in the art and described in more detail herein by way of example.

In some embodiments, the particles are structured such that the components calcium sulfate, an alkaline earth carbonate and gentamicin sulfate are present together in particles which are partially or completely surrounded by the binder within the particles. Accordingly, the composition according to the invention can comprise particles which comprise particles of calcium sulfate, an alkaline earth carbonate and gentamicin sulfate, wherein the particles are surrounded by a binder. The cover layer formed from the binder around the individual particles can be, for example, in each case below 100 μm thick. Such a structure can promote delayed release of the gentamicin sulfate, as described in more detail elsewhere herein.

In some embodiments, the particles have a substantially compact structure that is largely free of cavities. For example, the particles may be free of cavities having a diameter of more than 50 μm. In one embodiment, the composition comprises 60 to 90 percent by weight calcium sulfate. In some embodiments, the composition comprises 70 to 80 percent by weight calcium sulfate, for example 70 to 75 percent by weight, 75 to 80 percent by weight, or 73 to 77 percent by weight calcium sulfate.

In some embodiments, the composition comprises 10 to 20 percent by weight alkaline earth carbonate, for example 12 to 18 percent by weight or 14 to 16 percent by weight alkaline earth carbonate. The alkaline earth carbonate can be, for example, magnesium carbonate or calcium carbonate. The calcium carbonate may be crystalline calcium carbonate. The calcium carbonate can be, for example, calcite, aragonite or dolomite.

In some embodiments, the calcium sulfate is calcium sulfate dihydrate (CAS No. 10101-41-4), calcium sulfate hemihydrate (CAS No. 10034-76-1) or anhydrous calcium sulfate (CAS No. 7778-18-9). In some embodiments, the calcium sulfate is calcium sulfate dihydrate. The calcium sulfate may be present as crystalline or amorphous salt.

In a further embodiment, the composition comprises 5 to 15 percent by weight binder, for example 7 to 12 percent by weight binder. The binder may be a hydrophobic binder. “Hydrophobic” herein means that the binder is not miscible with water. In some embodiments, the binder has a partition coefficient log P (octanol/water) of at least 1.0, at least 5.0, or at least 10.0 under standard conditions. It may be advantageous for the binder to have a melting point of at least 45° C. The binder can be, for example, a triglyceride or alkanol, wherein the alkanol preferably comprises at least 12 carbon atoms. In some embodiments, the alkanol comprises at least 13, 14, 15, or 16 carbon atoms.

Triglycerides comprise 3 carboxylic acids, frequently also called “fatty acids,” which are esterified together with a glycerol molecule. Examples of triglycerides comprise glycerol tripalmitate (CAS No. 555-44-2), glycerol tristearate (CAS No. 555-43-1) and glycerol trilaurate (CAS No. 538-24-9). In some embodiments, the binder is a saturated triglyceride, i.e., a triglyceride containing exclusively fatty acids with saturated hydrocarbon content. Such fatty acids do not contain any double bonds or triple bonds in the hydrocarbon content. In some embodiments, the triglyceride comprises three identical fatty acid residues. In some embodiments, the triglyceride comprises exclusively fatty acid residues having an even number of carbon atoms. In some embodiments, the triglyceride comprises exclusively linear, unbranched fatty acid residues. The compositions according to the invention can obtain a single triglyceride or a mixture of different triglycerides, for example a mixture of glycerol tripalmitate and glycerol tristearate.

In some embodiments, the calcium sulfate is selected from the group consisting of calcium sulfate dihydrate (CAS No. 10101-41-4), calcium sulfate hemihydrate (CAS No. 10034-76-1) and anhydrous calcium sulfate (CAS No. 7778-18-9). In one embodiment, the calcium sulfate is calcium sulfate dihydrate.

Described herein are compositions for use in a therapeutic procedure. The composition described herein can be used as a bone substitute material in such a therapeutic procedure. For this purpose, the composition described herein can be introduced into a bone defect. For example, a bone defect can be filled with the composition described herein. The bone defect can be a bone cavity.

In some embodiments, the therapeutic procedure comprises filling a bone defect caused by a trauma, tumor or infection with the composition. Bone defects caused by trauma can occur due to the action of mechanical forces on the skeletal system, wherein due to the impact of force the bone tissue can break and can also split into bone fragments in the case of very great impact of force. Examples of bone defects include comminuted fractures, which are often cleared out surgically so that bone cavities remain in place of the bone fragments. If said bone cavities exceed a critical size (known as a critical size defect), it is no longer possible to close the bone cavity by spontaneous bone reformation without the support of suitable filling materials. “Critical-size bone defects” are defined in the art and herein as those that can no longer spontaneously heal during the life of a patient according to the medical diagnosis. These bone cavities can be filled with a composition described herein, optionally also with mixtures thereof with blood and/or allogeneic or autologous cancellous bone, or allogeneic or autologous bone marrow concentrate. Accordingly, in some embodiments, the therapeutic procedure comprises filling a bone defect, for example a bone defect caused by trauma, with a composition described herein, wherein the bone defect has a critical size.

Bone marrow concentrate is also referred to as purified bone marrow cells or bone marrow aspirate concentrate (BMAC). It can be obtained from primary bone marrow by filtration and centrifugation. A procedure for obtaining bone marrow concentrate is described, for example, in “Chahla, Jorge et al. “Bone Marrow Aspirate Concentrate Harvesting and Processing Technique.” Arthroscopy techniques vol. 6,2 e441-e445. 10 Apr. 2017, doi: 10.1016/j.eats.2016.10.024″. Accordingly, purified bone marrow cells contain a higher concentration of stem cells than primary bone marrow. In particular, such a preparation may include growth factors such as interleukin-1 receptor antagonist (IL-1RA) and mesenchymal stem cells as well as other progenitor cells.

Bone defects caused by a tumor can arise due to the surgical removal of bone tumors, for example osteosarcomas and enchondromas, or bone metastases. The resulting bone cavities can likewise be filled with the composition according to the invention, or mixtures thereof. The compositions described herein can also be used for filling surgically repaired bone cysts. Microbially induced inflammation of the bone tissue, which is sometimes also referred to as osteitis or osteomyelitis, can occur haematogenically or traumatically. Such inflammation is frequently treated by debridement comprising the surgical removal of necrotic and infected tissue. Such debridement regularly results in bone defects. Such bone defects produced by a debridement can also be treated with the compositions described herein and mixtures thereof. This treatment comprises, for example, filling the bone defects with a composition described herein, or a mixture of such a composition with viable cells, as explained in more detail elsewhere herein.

In accordance with the explanations presented above, the term “bone defects caused by trauma, tumor or infection” also comprises such bone defects which arise due to the surgical treatment of a condition induced by trauma, tumor or infection.

The patient to be treated may be a vertebrate, for example a mammal, or a human.

The compositions described herein may have a delayed release of active ingredient with respect to the gentamicin sulfate contained. This means that the gentamicin sulfate is continuously dispensed to an aqueous outer medium over a longer period of time, for example over a period of several days up to two weeks. These compositions can serve as antimicrobial active ingredient depots, in the case of which it is desirable for the local dispensing of an antimicrobial active ingredient, such as gentamicin sulfate, to take place over a longer period of time. The compositions in question can also serve as a vehicle for the local administration of antimicrobial active ingredients to bone tissue, in particular in the region of bone cavities.

In the therapeutic procedures described herein, the compositions described herein can be mixed with viable cells. For this purpose, such a composition can preferably be mixed with the cells outside the patient's body, i.e., in vitro. In some embodiments, a homogeneous mixture is thus obtained, i.e., the cells are present in a substantially uniform distribution within the mixture. In some embodiments, the viable cells form, with the composition according to the invention, from a macroscopic perspective a common phase in the mixture. The mixture thus obtained can be used as a bone substitute material. For example, the mixture can be introduced into a bone defect, in particular a bone cavity, of a patient, as described herein.

The viable cells are preferably cells which are suitable for supporting the healing of a bone defect. Examples of such cells are osteoblasts and osteocytes. For example, the compositions described herein can be mixed with blood or cancellous bone. When blood is used, it is preferable to use blood that is able to coagulate, for example whole blood. The viable cells can originate from the same patient to be treated with the mixture. These are also referred to as “autologous” cells. In some embodiments, cells from other donors can also be used. These are also referred to as “allogeneic” cells. Accordingly, the compositions described herein can be mixed with autologous or allogeneic blood, and/or with autologous or allogeneic cancellous bone.

If the patient to be treated is a human, the cells can preferably be human cells. In a corresponding manner, the cells provided in connection with the compositions described herein for use in the treatment of an animal patient can originate from the same species as the animal patient. The cancellous bone can be mixed in the form of bone chips with the composition described herein. Preferably, bone chips can be used which are 1 to 10 mm in length, for example 2 to 9 mm, 3 to 8 mm, 4 to 7 mm, 1 to 5 mm, or 2 to 4 mm in length. In order to determine this length, the maximum diameter of a bone chip is decisive in each case, wherein substantially all bone chips have a diameter in the range of, for example, 1 to 10 mm.

The composition used herein can be mixed, for example, in a weight ratio of “1 to 3” to “1 to 0.5,” with cancellous bone or bone marrow concentrate, i.e., a portion of the composition described herein is mixed with 0.5 to 3 parts (wt) of cancellous bone or 0.5 to 3 parts (wt) bone marrow concentrate, respectively.

In some embodiments, a composition described herein is mixed with blood in a weight ratio of “1 to 0.2” to “1 to 1,” i.e., a portion of the composition described herein is mixed with 0.2 to 1 parts blood. The blood can be, for example, whole blood. The blood is preferably coagulable. A mixture containing a composition described herein and coagulable blood may preferably be used after coagulation of blood for therapeutic application, in particular for filling a bone defect, as described herein.

In some embodiments, a composition described herein is mixed with both cancellous bone and blood. In this case, one part by weight of the composition according to the invention can preferably each be mixed with 0.5 to 3.0 parts by weight cancellous bone and with 0.2 to 0.8 parts by weight blood. The mixture formed thereby can then be subjected to coagulation of the blood contained therein. After blood coagulation has taken place, the mixture can be administered to the patient.

The viable cells, for example in the form of blood and/or cancellous bone, can be incorporated into the compositions according to the invention by hand, using a rotary mixer or using a pneumatic mixer.

In some embodiments in which the composition described herein is mixed with blood, the blood wets the surface of the composition after being mixed with the composition. As a result, the bonding to or the growth of patient-specific cells can be improved.

In some embodiments, the cells are mixed shortly after their removal from the donor with a composition described herein, and administered within a short time to the patient. For example, this administration can take place within 1 hour, or within 10, 20 or within 30 minutes after removal of the cells from the donor. In one embodiment, the removal of the cells from the donor and administration to the patient are performed in the same surgical procedure. In this case, the patient's own cells are removed from the patient by the attending physician, for example in the form of cancellous bone and/or blood, mixed with a composition according to the invention, and subsequently administered with the shortest possible time interval, optionally after coagulation of the blood, to the patient in the region of a bone defect.

The compositions according to the invention, as well as their mixtures with viable cells, can be administered by hand or by means of a suitable dispensing device, such as, for example, a manually operated piston discharge device or a syringe-like device, to a patient, in particular in the region of a bone defect such as, for example, a bone cavity.

When using a therapy method of the composition described herein, it is advantageous if the gentamicin sulfate content does not lead to cytotoxic effects. For example, preferably no cytotoxic effects should occur in the region of administration of the composition, for example within a bone defect. Such cytotoxic effects comprise, for example, the lysis of blood cells, such as, for example, leukocytes or erythrocytes, upon contact with a bone substitute material. This is particularly relevant when the composition is mixed with viable cells in vitro, as described above. A gentamicin sulfate content according to the invention can cause the viable cells to continue to maintain their ability to regenerate bone tissue after introduction into a bone cavity of a patient to be treated, which can lead to improved therapeutic success. For this purpose, a gentamicin sulfate content of not more than 2.5 percent by weight, based on the total weight of the composition according to the invention, can be advantageous. In some embodiments, the compositions described herein are non-cytotoxic according to the ISO 10993-5 standard. In some embodiments, the compositions described herein and the mixtures thereof described herein are non-cytotoxic according to the in-vitro cytotoxicity test of the ISO 10993-5 standard. In some embodiments, the compositions described herein and the mixtures thereof described herein are non-cytotoxic according to the acute systemic toxicity test according to the ISO 10993-5 standard.

Microbial colonization of bone substitute material may also affect the ability to regenerate bone tissue. Due to its antimicrobial effect, an effective concentration of gentamicin sulfate can therefore contribute to improved therapeutic success of the bone substitute material according to the invention. The antimicrobial effect of the gentamicin sulfate can protect both the bone substitute material and its site of use in the patient against colonization by microbial germs. The delayed release described herein can also contribute to improved therapeutic success by preventing, over a prolonged period, microbial colonization and infections that potentially result therefrom.

In some embodiments, the compositions or mixtures thereof described herein have the ability to serve as a suitable carrier material on which bone tissue can grow.

In some embodiments, the compositions or mixtures thereof described herein have an osteogenic effect. The term “osteogenic” refers here to the ability of a material to promote and/or accelerate growth of new bone tissue by one or more mechanisms, such as osteogenesis, osteoconduction and/or osteoinduction.

In addition to the above-mentioned components, the compositions according to the invention can also comprise a further therapeutic substance, for example, active ingredients for the induction of bone growth, such as bone morphogenetic proteins, growth factors, peptides and the like, antiviral agents, antibiotics, etc.); or monofile or multifile structures, films, coatings, membranes (e.g., porous, microporous, resorbable, etc.), foams (e.g., open-cell or closed-cell), screw augmentations, skull reconstructions and/or combinations thereof. The therapeutic substance can be contained in the compositions according to the invention. The therapeutic substances may comprise, but are not limited to, other antibiotics, chemotherapeutic agents, growth factors (in particular osteoinductive growth factors), such as bone morphogenetic proteins, endothelial growth factors, insulin growth factors, or the like, or a combination thereof. Non-limiting examples of usable further antimicrobial active ingredients are: antiamebics, e.g. arsthinol, bialamicol, carbarsons, cefaelins, chlorobetamides, chloroquin, chlorphenoxamides, chlortetracyclin, dehydroemetins, dibromopropamidins, diloxanides, diphetarsons, emetins, fumagillin, glaucarubin, glycobiarsol, 8-hydroxy-7-iodo-5-quinoline-sulfonic acid, iodochlorhydroxy quin, iodoquinol, paromomycin, phanquinone, poly benzarsol, propamidine, quinfamide, scenidazole, sulfarside, teclozan, tetracyclin, thiocarbamizin, thiocarbarsons, tinidazole: antibiotics, e.g., aminoglycosides (such as amikacin, apramycin, arbekacin, bambermycins, butirosin, dibekacin, dihydrostreptomycin, fortimicin (s), isepamicin, kaniamycin, micronomicin, neomycin, neomycin undecylenate, netilmicin, paromomycin, ribostamycin, sisomicin, spectinomycin, streptomycin, tobramycin, trospectomycin), amphenicols (azidamfenicol, chloramphenicol, florfenicol, thiamphenicol), ansamycins (rifamid, rifampin, rifamycin, rifapentin, rifaximin), lactams (carbacephems, loracarbef, carbapenem (biapenem, imipenem, meropenem, panipenem), cefalosporins (cefaclor, cefadroxil, cefamandol, cefatrizin, cefazedon, cefazolin, cefcapen povoxil, cefclidin, cefdinir, cefditoren, cefepime, cefetamet, cefixime, cefinenoxin, cefodizim, cefonicide, cefoperazone, ceforanide, cefotaxime, cefotiam, cefozopran, cefpimizol, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cefacetrile sodium, cefalexin, cefaloglycin, cefaloridine, cefalosporin, cefalotin, cefapirin sodium, cefradine, pivcefalexin), cefamycins (cefbuperazone, cefmetazole, cefminox, cefotetan, cefoxitin), monobactams (aztreonam, carumonam, tigemonam), oxacephens (flomoxef, moxalactam), penicillins (amdinocillin, amdinocillin pivoxil, amoxicillin, ampicillin, apalcillin, aspoxicillin, azidocillin, azlocillin, bacampicillin, benzyl penicillic acid, benzyl penicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamin, penimepicyclin, phenethicillin potassium, piperacillin, pivampicillin, propicillin, quinacillin, sulbenicillin, sultamicillin, talampicillin, temocillin, ticarcillin), ritipenem), lincosamides (clindamycin, lincomycin), makrolides (azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, eucomycins, midecamycins, miokamycin, oleandomycin, primycin, rokitamycin, rosaramicin, roxithromycin, spiramycin, troleandomycin), polypeptides (amphomycin, bacitracin, capreomycin, colistin, enduracidin, enviomycin, fusafungine, gramicidin S, gramicidin (s), mikamycin, polymyxin, pristinamycin, ristocetin, teicoplanin, thiostrepton, tuberactinomycin, tyrocidin, tyrothricin, vancomycin, viomycin, virginiamycin, zinc bacitracin), tetracyclines (apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), cycloserine, mupirocin, tuberin: synthetic antibacterial agents, e.g., 2,4-diaminopyrimidine (brodimoprim, textroxoprim, trimethoprim), nitrofuranes (furaltadon, furazoliumchloride, nifuradene, nifuratel, nifurfoline, nifurpirinol, nifurprazin, nifurtoinol, nitrofirantoin), quinolones and analogues (cinoxacin, ciprofloxacin, clinafloxacin, difloxacin, enoxacin, fleroxacin, flumequin, grepafloxacin, lomefloxacin, miloxacin, nadifloxacin, nadilixic acid, norflaxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (acetyl sulfamethoxpyrazine, benzyl sulfamide, chloramine-B, chloramine-T, dichloramine-T, N2-formyl sulfisomidine, N4-beta-D-glucosyl sulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanole, sulfalene, sulfaloxic, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, N4-sulfanilylsulfanilamide, sulfanilylurea, n-sulfanilyl-3,4-xylamide, sulfanitran, sulfaperin, sulfaphenazole, sulfaproxylin, sulfapyrazine, sulfapyridine, sulfasomizole, sulfasymazine, sulfathiazole, sulfathiourea, sulfatolamide, sulfisomidine, sulfisoxazole), sulfones (acedapsone, acediasulfone, acetosulfone sodium, dapsone, diathymosulfone, glucosulfone sodium, solasulfone, succisulfone, sulfanilic acid, p-sulfanilylbenzylamine, sulfoxon sodium, thiazolsulfone), clofoctol, hexedine, methenamine, methenamine anhydromethylencitrate, methenamine hippurate, methenamine mandelate, methenamine sulfosalicylate, nitroxoline, taurolidine, xibomole; leprostatic antibacterial agents, such as acedapsone, acetosulfone sodium, clofazimine, dapsone, diathymosulfone, glucosulfone sodium, hydnocarpinic acid, solasulfone, succisulfone, sulfoxone sodium, antifungals, such as allylamines, butenafine, naftifine, terbinafine, imidazoles (e.g., bifonazole, butoconazole, cholordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamate (tolcilate, tolindate, tolnaftate), triazolea (fluconazole, itraconazole, saperconazole, terconazole), acrisorcin, amorolfin, biphenamine, bromosalicylchloranilide, buclosamide, calcium propionate, chlorphenesine, ciclopirox, cloxyquin, coparaffinate, diamthazoldihydrochloride, exalamide, flucytosine, halethazole, hexetidine, loflucarbane, nifuratel, potassium iodide, propionic acid, pyrithione, salicylanilide, sodium propionate, sulbentin, tenonitrozole, triacetin, ujothione, undecylenic acid, zinc propionate; and the like.

Other further antimicrobial agents that are useful for the present invention comprise p-lactamase inhibitors (e.g., clavulanoic acid, sulbactam, tazobactam): chloramphenicols (e.g., azidamhenicol, chloramphenicol, thiaphenicol): fusidinic acid: synthetic agents, such as trimethoprine, optionally in combination with sulfonamides) and nitroimidazoles (e.g., metronidazole, tinidazole, nimorazole): antifungal agents (e.g., capreomycin, clofazimine, dapsone, ethambutol, isoniazid, pyrazinamide, rifabutin, rifampicin, streptomycin, thioamides); antiviral agents (e.g., acyclovir, amantadine, azidothymidine, ganciclovir, idoxuridine, tribavirin, trifluridine, vidarabine); interferons (e.g., interferon a, interferon p); and antiseptics (e.g., chlorhexidine, gentian violet, octenidine, povidone iodine, quaternary ammonium compounds, silver sulfadiazine, triclosan).

The further antimicrobial agent may comprise agents that treat diseases caused by gram-positive and/or gram-negative bacteria. Preferred antimicrobial agents include amikacin, tobramycin, vancomycin and salts thereof.

The therapeutic substance may further include a biological therapeutic substance, such as a protein. Proteins associated with bone can be added to alter the physical properties of the composition, to promote resorption, angiogenesis, cell entry and proliferation, mineralization, bone formation, growth of osteoclasts and/or osteoblasts, or the like. Proteins of particular interest are the various types of collagen, in particular type I. Other usable proteins are osteonectin, bone sialoproteins (Bsp), alpha-2HS-glycoproteins, bone Gla protein (Bgp), matrix Gla protein, bone phosphoglycoprotein, bone phosphoprotein, bone proteoglycan, protolipids, morphogenic bone proteins (e.g., BMP-1, -2A, -2B, -3, -3b, -4, -5, -6, -7, -8, -8b, -9, -10, -11, -12, -13, -14, -15), cartilage induction factor, platelet derived growth factor (PDGF-, -2), endothelial cell growth factor (ECGF-1, -2a, -2b), skeletal growth factor (IGF-2), insulin-like growth factors (IGF-1, IGF-2), fibroblast growth factor (ODGF-1,-2,-3,-4,-5,-6,-7,-8,-9,-10,-11,-12,-13,-14,-15, -16,-17,-18,-19,-20,-21, -22,-23), colony-stimulating factor, transforming growth factor (e.g. TGF-, B), vascular endothelial growth factors (VEGF), growth-/differentiation factors (GDF-1, -3, -5,-6,-7,-8,-9,-9B,-10,-11,-15,-16), osteogenic proteins (BMP-7, BMP-8, BMP-8b), brown growth hormone, parathormone (PTH), insulin, calcitonin and the like. The proteins can also include proteins associated with cartilage, such as the chondrocalcination protein, proteins associated with dentine, such as phosphophoryn, glycoproteins and Gla proteins, or proteins associated with enamel, such as amelognin and enamelin. The interesting structural proteins include fibrin, fibrinogen, keratin, tubulin, elastin and the like. Blood proteins can be used individually or together in plasma or serum, e.g., serum albumin.

The therapeutic substance may further comprise a non-protein growth factor, such as prostaglandins and statins (e.g., simvastatin, lovastatin).

In one embodiment, the therapeutic substance is a growth factor, such as bone morphogenetic proteins, endothelial cell growth factors, insulin-like growth factors, or the like, or a combination thereof.

Preferably, the further therapeutic substance is optionally used in a non-cytotoxic amount in the composition described herein.

A further aspect relates to a method for producing a composition, comprising the following steps:
grinding calcium sulfate, an alkaline earth carbonate, a binder, and more than 0 to 2.5 percent by weight gentamicin sulfate, for example 0.5 to 2.5 percent by weight gentamicin sulfate, based on the total mass of the composition, to a powder:
compacting the powder to form an agglomerate:
breaking down the agglomerate to a granulate; and
fractionating the granulate to a desired particle size.

The starting materials can in principle be present in any solid form which can be subjected to grinding. During grinding, grinding bodies can preferably be used, for example grinding balls which can consist, for example, of ceramic materials, such as porcelain or corundum. As a result of grinding, a powder of the above-mentioned starting materials is obtained. This powder preferably constitutes a homogenized mixture of said starting materials. The powder obtained in this way can then be compacted. Compacting can take place by any method that is customary and suitable in the art, for example with conventional rotary tableting presses or eccentric presses. Compacting can take place, for example, by pressing the powder with a force of at least 30 kN. The compacting results in a compact shaped body, such as a tablet, being obtained from the powder. Such a shaped body can have a regular shape, for example the shape of a cylinder, a sphere or an ovoid. Such a shaped body is also referred to herein as “agglomerate.” An example of an agglomerate is a tablet which can be produced by pressing a powder.

Granulate can be obtained by breaking down the agglomerate. The agglomerate can be broken down, for example, by means of a crushing sieve or a sieve mill, as known and customary in the art. The individual granules (i.e., grains) of such a granulate frequently have a random shape and size. The granulate can, for example, be fractionated using a sieve tower, as is customary in the art and described in the examples below. In this way, a granulate having a desired particle size fraction can be obtained. The desired particle size is, for example, 1 to 7 mm, as described in more detail elsewhere herein.

Preferably, the method is carried out under conditions which avoid melting of the binder. For this purpose, for example, work can be carried out under ambient conditions below the melting temperature of the binder, and/or with the process steps mentioned, in particular the grinding, the heat generation can be monitored and controlled accordingly.

One aspect relates to a granulate which can be produced according to the method described above. The granulate can comprise calcium sulfate, an alkaline earth carbonate, a binder, and 0.5 to 2.5 percent by weight gentamicin sulfate, based on the total mass of the composition.

A further aspect relates to an agglomerate comprising calcium sulfate, an alkaline earth carbonate, a binder, and more than 0 to 2.5 percent by weight gentamicin sulfate, based on the total mass of the composition. The agglomerate may preferably comprise 0.5 to 2.5 percent by weight gentamicin sulfate, for example 1.2 to 2.0; 1.4 to 1.8%; or 1.5 to 1.7%, or 1.55% to 1.65%.

The agglomerates can, as described previously in more detail herein, be produced by compacting a powder. The agglomerates can have, for example, a cylindrical shape having a height of 5 to 15 mm and a diameter of 15 to 40 mm. The agglomerates can also have a spherical or ovoid shape of comparable size.

The agglomerates may optionally be broken down into irregular shaped granules in order to obtain a bone substitute material described herein.

In some embodiments, the compositions or agglomerates described herein contain, for example, 1.6 wt % gentamicin sulfate. This means that, with an activity coefficient of AK=600 of the gentamicin sulfate, 1.0 percent by weight gentamicin base is contained in the composition or the compacted mixture. An activity coefficient of 600 means that 600 μg gentamicin base is present in 1 mg gentamicin sulfate.

A further aspect of the invention relates to a composition comprising calcium sulfate, an alkaline earth carbonate, a binder, more than 0 to at most 2.5 percent by weight gentamicin sulfate, based on the total mass of the composition.

A second embodiment of this aspect relates to a composition comprising particles in which the calcium sulfate, the binder, the alkaline earth carbonate and the gentamicin sulfate are present together.

In this case, the particles can have an irregular shape which can be produced by breaking down agglomerates.

The particles can have an average diameter of 1.0 to 7.0 mm, wherein the average diameter of the particles can be determined by fractionated screening.

The composition may comprise, for example, 70 to 80 percent by weight calcium sulfate.

The composition may comprise 10 to 20, or preferably 12 to 18 percent by weight alkaline earth carbonate.

The composition may comprise 5 to 15, preferably 7 to 12 percent by weight binder.

The binder may have a melting point of at least 45° C.

The binder may be a triglyceride or alkanol, wherein the alkanol preferably comprises at least 12 carbon atoms.

The calcium sulfate may be selected from the group consisting of calcium sulfate dihydrate, calcium sulfate hemihydrate, and anhydrous calcium sulfate.

The alkaline earth carbonate can be selected from the group consisting of calcite, aragonite, magnesium carbonate and dolomite.

A further aspect of the invention relates to a therapeutic procedure in which a composition described herein is administered to a patient as described herein.

### EXAMPLES

The invention is further illustrated below using examples which are, however, not to be understood as limiting. It will be apparent to a person skilled in the art that other equivalent means may be used similarly in place of the features described here.

For the following examples, calcium sulfate dihydrate (CS), calcium carbonate (CC), magnesium carbonate (MC), glycerol tripalmitate (GTP), glycerol tristearate (GTS) gentamicin sulfate ((GS), activity coefficient AC=600) from Sigma-Aldrich was used. The glycerol tripalmitate (GTP) was used as a fine powder having a particle size of significantly less than 1 mm.

### EXAMPLES 1 TO 7: PREPARATION OF BONE SUBSTITUTE MATERIALS

The mixtures of the examples 1-7 shown in the table were homogenized within 10 hours with a Turbula mixer using porcelain milling balls. Subsequently, the homogenized mixtures were pressed with an eccentric tableting press to form cylindrical shaped bodies having a height of h=10 mm, a diameter of 25 mm. The cylindrical shaped bodies were comminuted with an oscillating Frewitt crushing sieve and the granulate obtained was fractionated into 1-3 mm and 3-5 mm fractions using a sieve tower. The weight ratio of calcium sulfate dihydrate to calcium carbonate/magnesium carbonate to glycerol tripalmitate/glycerol tristearate was kept constant and the content of gentamicin sulfate varied between 0.5 percent by weight and 2.5 percent by weight.

TABLE 1
Composition of the mixtures in examples 1-6. CS: calcium
sulfate dihydrate, CC: calcium carbonate, MC: magnesium
carbonate, GTP: glycerol tripalmitate, GTS: glycerol tristearate,
GS: gentamicin sulfate, GB: gentamicin base.

| | Weight of sample [g] | | | | | | |
|---|---|---|---|---|---|---|---|
| Example | CS | CC | MC | GTP | GTS | GS (GB) | |
| 1 | 361.0 | 90.3 | — | 44.5 | — | 4.2 | 0.84 wt. % GS |
| | | | | | | 2.5 | 0.50 wt. % GB |
| 2 | 358.0 | 89.4 | — | 44.2 | — | 8.4 | 1.68 wt. % GS |
| | | | | | | 5.0 | 1.00 wt. % GB |
| 3 | 355.0 | 88.7 | — | 43.8 | — | 12.5 | 2.50 wt. % GS |
| | | | | | | | 1.50 wt. % GB |
| 4 | 351.8 | 88.0 | — | 43.5 | — | 16.7 | 3.34 wt. % GS |
| | | | | | | | 2.00 wt. % GB |
| 5 | 348.9 | 87.2 | — | 43.1 | — | 20.8 | 4.16 wt. % GS |
| | | | | | | | 2.50 wt. % GB |
| 6 | 358.0 | — | 89.4 | 44.2 | — | 8.4 | 1.68 wt. % GS |
| | | | | | | 5.0 | 1.00 wt. % GB |
| 7 | 358.0 | 89.4 | — | — | 44.2 | 8.4 | 1.68 wt. % GS |
| | | | | | | 5.0 | 1.00 wt. % GB |

The in vitro cytotoxicity of the test specimens of Examples 1-6 was tested according to ISO 1099-5. The test specimens of Examples 1-3, 6 and 7 were non-cytotoxic. The test specimens of Examples 4 and 5 demonstrated a cytotoxic effect.

In the following examples, the miscibility of the bone substitute material according to the invention with whole blood and with allogeneic bone chips (allogeneic cancellous bone) was tested.

### Example 8

5.0 g granulate of the 1-3 mm fraction of Example 2 was mixed with 1.0 ml (˜1.0 g) fresh full blood (porcine blood) in a kidney dish. After around 10 minutes, a gel-like mass was obtained after coagulation of the whole blood.

### Example 9

5.0 g granulate of the 1-3 mm fraction of Example 2 was mixed with 5.0 ml (˜5.0 g) fresh whole blood (porcine blood) in a kidney dish. After around 10 minutes, a gel-like mass was obtained after coagulation of the whole blood.

### Example 10

5.0 g granulate of the 1-3 mm fraction of Example 2 was mixed with 2.5 g allogeneic bone chips (size of the chips 2-4 mm). A homogeneous mixture was produced.

### Example 11

5.0 g granulate of the 1-3 mm fraction of Example 2 was mixed with 15.0 g allogeneic bone chips (size of the chips 2-4 mm). A homogeneous mixture was likewise produced.

### Example 12

5.0 g granulate of the 1-3 mm fraction of Example 2 was mixed with 5.0 g allogeneic bone chips (size of the chips 2-4 mm) and with 5.0 ml (˜ 5.0 g) fresh whole blood (porcine blood). A homogeneous mixture was likewise produced.

1. A composition for use in a therapeutic procedure, wherein the composition comprises:
calcium sulfate;
an alkaline earth carbonate;
a binder; and
more than 0 to at most 2.5 percent by weight gentamicin sulfate.

2. The composition of claim 1, further comprising 0.5 to 2.5 percent by weight gentamicin sulfate, based on the total mass of the composition

3. The composition of claim 1, wherein a homogeneous mixture is formed by mixing the composition with viable cells in vitro.

4. The composition of claim 3, wherein the viable cells are in the form of whole blood, or are in the form of bone marrow concentrate, or are in the form of cancellous bone, and are mixed in vitro with the composition.

5. The composition of claim 4, wherein the viable cells are autologous or an allogeneic.

6. The composition of claim 4, wherein the cancellous bone is in the form of bone chips.

7. The composition of claim 6, wherein the bone chips have a mean diameter of 1 to 10 mm.

8. The composition of claim 4, wherein the composition is mixed with cancellous bone or bone marrow concentrate in vitro in a weight ratio of 1 to 0.5 to 1 to 3.

9. The composition of claim 4, wherein the composition is mixed with blood in vitro in a weight ratio of 1 to 0.2 to 1 to 1.

10. The composition of claim 4, wherein each part by weight of the composition is mixed in vitro with 0.5 to 3.0 parts by weight cancellous bone or bone marrow concentrate and with 0.2 to 0.8 parts by weight blood.

11. The composition of claim 4, wherein the blood is subjected to coagulation after being mixed with the composition.

12. The composition of claim 4, wherein the composition is introduced into a bone defect or a bone cavity.

13. The composition of claim 4, wherein the composition comprises 70 to 80 percent by weight calcium sulfate.

14. The composition of claim 4, wherein the composition comprises 12 to 18 percent by weight alkaline earth carbonate.

15. The composition of claim 4, wherein the composition comprises 7 to 12 percent by weight binder.

16. The composition of claim 4, wherein the therapeutic procedure comprises filling a bone defect caused by a trauma, tumor or infection with the composition.

17. The composition of claim 4, wherein the composition comprises particles having an irregular shape, and said particles are obtained by breaking an agglomerate.

18. A method of treatment of bone defects comprising:
filling the bone defects with a composition, the composition comprising:
calcium sulfate;
an alkaline earth carbonate;
a binder; and
more than 0 to at most 2.5 percent by weight gentamicin sulfate.

19. The method of claim 18, further comprising mixing the composition with viable cells in vitro, thereby forming a homogeneous mixture.

20. The method of claim 19, wherein the viable cells in the form of blood, preferably whole blood, and/or in the form of bone marrow concentrate, and/or in the form of cancellous bone, are mixed in vitro with the composition.

21. The method of claim 19, further comprising, prior to filling the bone defects with a composition, debridement of bone comprising the surgical removal of necrotic and infected tissue, thereby at least partially causing the bone defects.

21. The method of claim 19, wherein the composition comprises particles having a mean particle diameter of 1.0 to 7.0 mm, 0.5 to 10 mm, 1 to 3 mm, 2 to 6 mm, 3 to 5 mm, or 3.5 to 4.5 mm.

22. The method of claim 19, wherein the composition comprises particles having an irregular shape, and said particles are obtainable by breaking an agglomerate.
